(19) United States
(12) Patent Application Publication
(10) Pub. No.: US 2003/0109525 A1
(43) Pub. Date: Jun. 12, 2003

(54) Crystalline thiazine oxazolidinones

(75) Inventors: Meredith L. Greene, Kalamazoo, MI (US); Jiong J. Chen, Kalamazoo, MI (US); Mark T. Maloney, Kalamazoo, MI (US); Cuong V. Lu, Kalamazoo, MI (US)

Correspondence Address: MUETING, RAASCH & GEBHARDT, P.A., P.O. BOX 581415, MINNEAPOLIS, MN, 55458, US

(73) Assignee: PHARMACIA & UPJOHN COMPANY, Kalamazoo, MI (US)

(21) Appl. No.: 10/193,765

(22) Filed: Jul. 10, 2002

Related U.S. Application Data
(60) Provisional application No. 60/304,157, filed on Jul. 10, 2001.

(51) Int. Cl. A61K 31/541; C07D 417/02

(52) U.S. Cl. 514/227.8; 544/60

(57) ABSTRACT

The present invention provides crystals including a thiazine oxazolidinone. Thiazine oxazolidinones are useful as antimicrobials.

This application claims the benefit of the U.S. Provisional Application Serial No. 60/304,157, filed Jul. 10, 2001, which is incorporated herein by reference in its entirety.

### FIELD OF THE INVENTION

The present invention relates to crystals of N-({(5S)-3-[4-(1,1-dioxido-4-thiomorpholinyl)-3,5-difluorophenyl]-2-oxo-1,3-oxazolidin-5-yl}methyl) acetamide and methods for isolation of such crystals.

### BACKGROUND

Oxazoidinone antibacterial agents are a novel synthetic class of antimicrobials with potent activity against a number of human and veterinary pathogens, including gram-positive aerobic bacteria such as multiply-resistant staphylococci and streptococci, gram-negative aerobic bacteria such as H. influenzae and M. catarrhalis, as well as anaerobic organisms such as bacteroides and clostridia species, and acid-fast organisms such as Mycobacterium tuberculosis and Mycobacterium avium. It is also known that as a chemical compound class, oxazolidinones generally inhibit to some extent monoamine oxidase (MAO), the enzyme responsible for preventing acute blood pressure elevation by the endogenous and dietary amine, tyramine, and other sympathomimetic amines. Accordingly, there is a demand to discover oxazolidinone antibiotics which possess minimum MAO inhibitory activity to eliminate the related side effects from potential drug-drug interactions. There also exists a need for oxazolidinone antibiotics having improved antimicrobial activity, low toxicity, and adequate stability.

N-({(5S)-3-[4-(1,1-dioxido-4-thiomorpholinyl)-3,5-difluorophenyl]-2-oxo-1,3-oxazolidin-5-yl}methyl)acetamide is a thiazine oxazolidinone having the following structure:

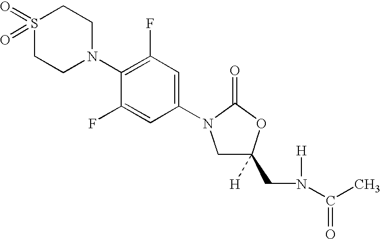

N-({(5S)-3-[4-(1,1-dioxido-4-thiomorpholinyl)-3,5-difluorophenyl]-2-oxo-1,3-oxazolidin-5-yl}methyl)acetamide is disclosed in PCT International Publication Number WO 01/98297 (Barbachyn et al.). Although the preparation of the thiazine oxazolidinone is disclosed, the specification is silent as to the isolation and nature of crystal forms of the agent. Moreover, there exists a need for crystalline forms of such materials that have superior chemical and/or physical properties that are useful in drug delivery applications.

### SUMMARY OF THE INVENTION

In one aspect, the present invention provides an anhydrous crystal including N-({(5S)-3-[4-(1,1-dioxido-4-thiomorpholinyl)-3,5-difluorophenyl]-2-oxo-1,3-oxazolidin-5-yl}nethyl)acetamide, which has the structure:

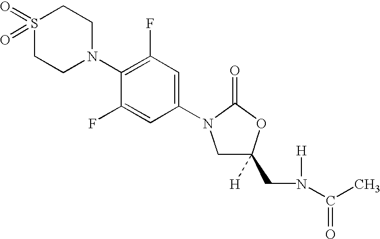

Preferably the crystal has a melting point of at least about 170° C., more preferably at least about 180° C., and most preferably at least about 187° C. Preferably the thiazine oxazolidinone is stable in a bulk drug stability test at 56° C. and 75% relative humidity for at least 25 days, and more preferably at 70° C. and 75% relative humidity for at least 60 days.

In another aspect, the present invention provides a crystal including N-({(5S)-3-[4-(1,1-dioxido-4-thiomorpholinyl)-3,5-difluorophenyl]-2-oxo-1,3-oxazolidin-5-yl}methyl)acetamide including at most about 1% by weight water, preferably at most about 0.5% by weight water, and most preferably at most about 0.1% by weight water. Preferably, the N-({(5S)-3-[4-(1,1-dioxido-4-thiomorpholinyl)-3,5-difluorophenyl]-2-oxo-1,3-oxazolidin-5-yl}-methyl)acetamide is stable in a bulk drug stability test at 56° C. and 75% relative humidity for at least 25 days, and more preferably at 70° C. and 75% relative humidity for at least 60 days.

In another aspect, the present invention provides a crystal including N-({(5S)-3-[4-(1,1-dioxido-4-thiomorpholinyl)-3,5-difluorophenyl]-2-oxo-1,3-oxazolidin-5-yl}methyl)acetamide having orthorhombic space group symmetry P2₁2₁2₁.

In another aspect, the present invention provides a crystal including N-({(5S)-3-[4-(1,1-dioxido-4-thiomorpholinyl)-3,5-difluorophenyl]-2-oxo-1,3-oxazolidin-5-yl}methyl)acetamide including a unit cell defined by the dimensions a, b, c, α, β, and γ, wherein a is about 8.8 Å, b is about 11.5 Å, c is about 17.4 Å, and α=β=γ=90°.

In another aspect, the present invention provides a crystal including N-({(5S)-3-[4-(1,1-dioxido-4-thiomorpholinyl)-3,5-difluorophenyl]-2-oxo-1,3-oxazolidin-5-yl}lmethyl)acetamide having orthorhombic space group symmetry P2₁2₁2₁ and including a unit cell defined by the dimensions a, b, c, α, β, and γ, wherein a is about 8.8 Å, b is about 11.5 Å, c is about 17.4 Å, and α=β=γ=90°.

In another aspect, the present invention provides a crystal including N-({(5S)-3-[4-(1,1-dioxido-4-thiomorpholinyl)-3,5-difluorophenyl]-2-oxo-1,3-oxazolidin-5-yl}methyl)acetamide, wherein the atomic positions of the atoms of the N-({(5S)-3-[4-(1,1-dioxido-4-thiomorpholinyl)-3,5-difluorophenyl]-2-oxo-1,3-oxazolidin-5-yl}methyl)acetamide are defined by a set of points having a root mean square deviation of less than about 0.1 ÅA from the structure coordinates listed in Table 1. Preferably, the atomic positions of the atoms are defined by the structure coordinates listed in Table 1.

In another aspect, the present invention provides a crystal including N-({(5S)-3-[4-(1,1-dioxido-4-thiomorpholinyl)-3,5-difluorophenyl]-2-oxo-1,3-oxazolidin-5-yl} methyl)acetamide having characteristic diffraction peaks at about 21.9, 21,4, and 16.1 degrees two-theta in an X-ray powder diffraction pattern. Preferably, the crystal has characteristic diffraction peaks at about 29.5, 21.9, 21.4, 18.4, 16.1, and 9.2 degrees two-theta in an X-ray powder diffraction pattern. More preferably, the crystal has the characteristic diffraction peaks in an X-ray powder diffraction pattern as listed in Table 5.

In another aspect, the present invention provides a solvated crystal including N-({(5S)-3-[4-(1,1-dioxido-4-thiomorpholinyl)-3,5-difluorophenyl]-2-oxo-1,3-oxazolidin-5-yl}methyl)acetamide, wherein the solvate includes a solvent selected from the group consisting of tetrahydrofuran, acetone, ethyl acetate, acetonitrile, methanol, ethanol, propanol, isopropanol, and combinations thereof.

In another aspect, the present invention provides a crystal including N-({(5S)-3-[4-(1,1-dioxido-4-thiomorpholinyl)-3,5-difluorophenyl]-2-oxo-1,3-oxazolidin-5-yl}methyl)acetamide having characteristic diffraction peaks at about 18.9 and 6.3 degrees two-theta in an X-ray powder diffraction pattern. Preferably, the crystal has characteristic diffraction peaks at about 19.2, 18.9, 16.4, 9.6, and 6.3 degrees two-theta in an X-ray powder diffraction pattern. More preferably, the crystal has the characteristic peaks in an X-ray powder diffraction pattern as listed in Table 2, Table 3, Table 4, or combinations thereof.

In another aspect, the present invention provides a method of isolating an anhydrous crystal including thiazoline oxazolidinone including cooling an aqueous solution of a solvate including N-({(5S)-3-[4-(1,1-dioxido-4-thiomorpholinyl)-3,5-difluorophenyl]-2-oxo-1,3-oxazolidin-5-yl}-methyl)acetamide to give the anhydrous crystal including the thiazoline oxazolidinone. Preferably the solvate includes an organic solvent, and more preferably a non-halogenated solvent. Optionally, the aqueous solution further includes a water miscible organic solvent, and preferably a non-halogenated solvent.

In another aspect, the present invention provides a method of isolating a solvated thiazoline oxazolidinone including cooling a composition including N-({(5S)-3-[4-(1,1-dioxido-4-thiomorpholinyl)-3,5-difluorophenyl]-2-oxo-1,3-oxazolidin-5-yl}methyl)acetamide and an organic solvent selected from the group consisting of tetrahydrofuran, acetone, ethyl acetate, acetonitrile, methanol, ethanol, propanol, isopropanol, and combinations thereof, to give the solvated thiazoline oxazolidinone. Preferably, the medium further includes water.

In another aspect, the present invention provides a method of isolating an anhydrous crystal including N-({(5S)-3-[4-(1,1-dioxido-4-thiomorpholinyl)-3,5-difluorophenyl]-2-oxo-1,3-oxazolidin-5-yl}methyl)acetamide including inducing crystallization of N-({(5S)-3-[4-(1,1-dioxido-4-thiomorpholinyl)-3,5-difluorophenyl]-2-oxo-1,3-oxazolidin-5-yl}methyl)acetamide from a medium including methanol and methylene chloride.

Definitions

As referred to in the present application, “water miscible” means capable of being mixed with or dissolved in water at all proportions.

As referred to in the present application, “anhydrous crystalline” refers to a crystal that does not contain substantial amounts of water. The water content can be determined by methods known in the art including, for example, Karl Fischer titrations. Preferably an anhydrous crystalline form contains at most about 1% by weight water, more preferably at most about 0.5% by weight water, and most preferably at most about 0.1% by weight water.

As used herein, “solvate” and “solvated crystal” are used interchangeably and refer to a crystal that includes a solvent molecule.

As referred to in the present application, “stable” in bulk drug stability tests means that at least about 90% by weight, preferably at least about 95% by weight, and more preferably at least about 99% by weight of the bulk drug remains unchanged after storage under the indicated conditions for the indicated time.

### BRIEF DESCRIPTION OF THE FIGURES

FIG. 1 is an illustration of a ball and stick model representing the crystal structure coordinates of anhydrous crystalline N-({(5S)-3-[4-(1,1-dioxido-4-thiomorpholinyl)-3,5-difluorophenyl]-2-oxo-1,3-oxazolidin-5-yl}methyl)acetami de as listed in Table 1.

### DETAILED DESCRIPTION OF PREFERRED EMBODIMENTS

N-({(5S)-3-[4-(1,1-dioxido-4-thiomorpholinyl)-3,5-difluorophenyl]-2-oxo-1,3-oxazolidin-5-yl}methyl)acetamide is a thiazine oxazolidinone having the following structure:

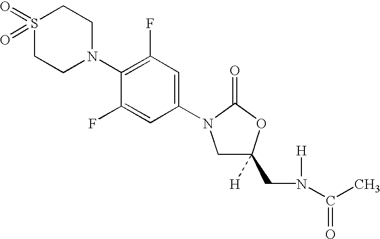

N-({(5S)-3-[4-(1,1-dioxido-4-thiomorpholinyl)-3,5-difluorophenyl]-2-oxo-1,3-oxazolidin-5-yl}methyl)acetamide can be prepared by standard synthetic procedures including, for example, those described in PCT International Publication Number WO 01/98297 (Barbachyn et al.), in which the thiazine oxazolidinone was isolated as a white solid, for example, by concentrating the thiazine oxazolidinone from a solution that included methanol and methylene chloride.

It has now been found that the thiazine oxazolidinone can be isolated as a white, crystalline, odorless solid in either a solvated or an anhydrous crystalline form. Solvated crystalline forms appear to undergo a conversion to an anhydrous crystalline form at about 100° C., thus providing a useful method for producing an anhydrous crystalline form.

The present invention provides intermediates and methods for isolating an anhydrous crystalline form of N-({(5S)-3-[4-(1,1-dioxido-4-thiomorpholinyl)-3,5-difluorophenyl]-2-oxo-1,3-oxazolidin-5-yl}methyl)acetamide. The anhydrous crystalline form shows improved properties in comparison to the solvated crystalline form.

Single Crystal X-ray Crystallographic Analysis

X-ray diffraction data was collected on a single crystal of the anhydrous crystalline form of N-({(5S)-3-[4-(1,1-dioxido-4thiomorpholinyl)-3,5-difluorophenyl]-2-oxo-1,3-oxazolidin-5-yl}methyl)acetamide, and the structure was solved by standard crystallographic techniques. The crystal had unit cell dimensions of a=8.7790 Å, b=11.4911 Å, c=17.4233 Å, and α=β=γ=90°.

Table 1 lists atomic structure coordinates derived by x-ray diffraction of a crystal of N-({(5S)-3-[4-(1,1-dioxido-4-thiomorpholinyl)-3,5-difluorophenyl]-2-oxo-1,3-oxazolidin-5-yl}methyl)acetamide having orthorhombic space group symmetry P2₁2₁2₁. Column 2 lists a number for the atom in the structure. Column 3 lists the element whose coordinates are measured. The first letter in the column defines the element. Columns 5-7 list the crystallographic coordinates X, Y, and Z, respectively. The crystallographic coordinates define the atomic position of the element measured. Column 8 lists an occupancy factor that refers to the fraction of the molecules in which each atom occupies the position specified by the coordinates. A value of “1” indicates that each atom has the same conformation, i.e., the same position, in all molecules of the crystal. Column 9 lists a thermal factor “B” that measures movement of the atom around its atomic center.

Each of the atoms of N-({(5S)-3-[4-(1,1-dioxido-4thiomorpholinyl)-3,5-difluorophenyl]-2-oxo-1,3-oxazolidin-5-yl}methyl)acetamide is defined by a set of structure coordinates as set forth in Table 1. The term “structure coordinates” refers to Cartesian coordinates derived from mathematical equations related to the patterns obtained on diffraction of a monochromatic beam of x-rays by the atoms (scattering centers) of a human beta secretase complex in crystal form. The diffraction data are used to calculate an electron density map of the repeating unit of the crystal. The electron density maps are then used to establish the positions of the individual atoms of the thiazine oxazolidinone.

Slight variations in structure coordinates can be generated by mathematically manipulating the thiazine oxazolidinone structure coordinates. For example, the structure coordinates set forth in Table 1 could be manipulated by crystallographic permutations of the structure coordinates, fractionalization of the structure coordinates, integer additions or subtractions to sets of the structure coordinates, inversion of the structure coordinates or any combination of the above. Alternatively, modifications in the crystal structure due to mutations, additions, substitutions, and/or deletions of atoms, or other changes in any of the components that make up the crystal, could also yield variations in structure coordinates. Such slight variations in the individual coordinates will have little effect on overall shape. If such variations are within an acceptable standard error as compared to the original coordinates, the resulting three-dimensional shape is considered to be structurally equivalent. Structural equivalence is described in more detail below.

It should be noted that slight variations in individual structure coordinates of the thiazine oxazolidinone would not be expected to significantly alter the activity of the compound. For the purpose of this invention, any molecule or molecular complex or any portion thereof, that has a root mean square deviation of conserved backbone atoms (C, N, O, S) of less than about 0.1 Å, when superimposed on the relevant backbone atoms described by the reference structure coordinates listed in Table 1, is considered “structurally equivalent” to the reference molecule. That is to say, the crystal structures of those portions of the two molecules are substantially identical, within acceptable error. Particularly preferred structurally equivalent molecules or molecular complexes are those that are defined by the entire set of structure coordinates listed in Table 1±a root mean square deviation from the conserved backbone atoms of those amino acids of less than about 0.1 Å. More preferably, the root mean square deviation is at most about 0.01 Å, and even more preferably, at most about 0.001 Å.

The term “root mean square deviation” means the square root of the arithmetic mean of the squares of the deviations. It is a way to express the deviation or variation from a trend or object. For purposes of this invention, the “root mean square deviation” defines the variation in the backbone of a compound from the backbone of the thiazine oxazolidinone defined by the structure coordinates described herein.

It will be readily apparent to those of skill in the art that the numbering of atoms in other isoforms of thiazine oxazolidinones may be different than that of N-({(5S)-3-[4-(1,1-dioxido-4-thiomorpholinyl)-3,5-difluorophenyl]-2-oxo-1,3-oxazolidin-5-yl}methyl)acetamide.

TABLE 1
Structural Coordinates for the Anhydrous Crystalline Form of the
Thiazine Oxazolidinone

| | | | | | | |
|---|---|---|---|---|---|---|
| CRYST1 | 8.779 | 11.491 | 17.423 | 90.00 | 90.00 | 90.00 |

| | | | | |
|---|---|---|---|---|
| SCALE1 | 0.113908 | 0.000000 | 0.000000 | 0.000000 |
| SCALE2 | 0.000000 | 0.087024 | 0.000000 | 0.000000 |
| SCALE3 | 0.000000 | 0.000000 | 0.057394 | 0.000000 |

| | | | | | | | | |
|---|---|---|---|---|---|---|---|---|
| ATOM | 1 | S1 | 0 | 1.128 | 6.484 | 3.992 | 1.000 | 2.86 |
| ATOM | 2 | F1 | 0 | 1.397 | 6.570 | −0.869 | 1.000 | 3.15 |
| ATOM | 3 | F2 | 0 | −0.168 | 10.659 | 0.838 | 1.000 | 2.96 |
| ATOM | 4 | O1 | 0 | 2.142 | 5.686 | 4.635 | 1.000 | 4.65 |
| ATOM | 5 | O2 | 0 | −0.102 | 6.732 | 4.699 | 1.000 | 3.72 |
| ATOM | 6 | O3 | 0 | 1.030 | 12.545 | −3.335 | 1.000 | 3.28 |
| ATOM | 7 | O4 | 0 | 0.261 | 9.556 | −9.028 | 1.000 | 2.84 |
| ATOM | 8 | O5 | 0 | 1.102 | 11.767 | −5.435 | 1.000 | 2.64 |
| ATOM | 9 | N1 | 0 | 0.692 | 7.983 | 1.363 | 1.000 | 2.96 |
| ATOM | 10 | N2 | 0 | 2.176 | 9.237 | −7.922 | 1.000 | 2.51 |
| ATOM | 11 | H2 | 0 | 2.809 | 8.684 | −7.740 | 1.000 | 3.01 |
| ATOM | 12 | N3 | 0 | 0.943 | 10.278 | −3.803 | 1.000 | 2.26 |
| ATOM | 13 | C1 | 0 | 1.029 | 11.609 | −4.095 | 1.000 | 2.38 |
| ATOM | 14 | C2 | 0 | 0.427 | 10.493 | −1.412 | 1.000 | 2.37 |
| ATOM | 15 | H2 | 0 | 0.221 | 11.393 | −1.524 | 1.000 | 2.85 |
| ATOM | 16 | C3 | 0 | 1.079 | 7.865 | −1.038 | 1.000 | 2.38 |
| ATOM | 17 | C4 | 0 | 0.317 | 9.905 | −0.181 | 1.000 | 2.48 |
| ATOM | 18 | C5 | 0 | 1.683 | 7.795 | −9.789 | 1.000 | 3.44 |
| ATOM | 19 | H5A | 0 | 0.994 | 7.678 | −10.447 | 1.000 | 5.16 |
| ATOM | 20 | H5B | 0 | 1.777 | 6.987 | −9.278 | 1.000 | 5.16 |
| ATOM | 21 | H5C | 0 | 2.514 | 7.994 | −10.227 | 1.000 | 5.16 |
| ATOM | 22 | C6 | 0 | 1.016 | 10.480 | −6.106 | 1.000 | 2.40 |
| ATOM | 23 | H6A | 0 | 0.137 | 10.382 | −6.528 | 1.000 | 2.88 |
| ATOM | 24 | C7 | 0 | 1.170 | 9.455 | −4.984 | 1.000 | 2.51 |
| ATOM | 25 | H7A | 0 | 0.510 | 8.749 | −5.056 | 1.000 | 3.01 |
| ATOM | 26 | H7B | 0 | 2.058 | 9.065 | −4.979 | 1.000 | 3.01 |
| ATOM | 27 | C8 | 0 | 1.147 | 8.397 | −2.306 | 1.000 | 2.35 |
| ATOM | 28 | H8A | 0 | 1.389 | 7.863 | −3.027 | 1.000 | 2.82 |
| ATOM | 29 | C9 | 0 | −0.093 | 6.783 | 1.594 | 1.000 | 2.72 |
| ATOM | 30 | H9A | 0 | −0.325 | 6.376 | 0.745 | 1.000 | 3.27 |
| ATOM | 31 | H9B | 0 | −0.915 | 7.013 | 2.054 | 1.000 | 3.27 |
| ATOM | 32 | C10 | 0 | 0.852 | 9.728 | −2.494 | 1.000 | 2.16 |
| ATOM | 33 | C11 | 0 | 0.674 | 8.585 | 0.094 | 1.000 | 2.50 |
| ATOM | 34 | C12 | 0 | 0.931 | 8.792 | 2.553 | 1.000 | 2.96 |
| ATOM | 35 | H12 | 0 | 0.089 | 8.993 | 2.989 | 1.000 | 3.55 |
| ATOM | 36 | H12 | 0 | 1.351 | 9.630 | 2.301 | 1.000 | 3.55 |
| ATOM | 37 | C13 | 0 | 1.838 | 8.027 | 3.509 | 1.000 | 2.79 |
| ATOM | 38 | H13 | 0 | 1.998 | 8.567 | 4.299 | 1.000 | 3.35 |
| ATOM | 39 | H13 | 0 | 2.693 | 7.866 | 3.079 | 1.000 | 3.35 |
| ATOM | 40 | C14 | 0 | 1.317 | 8.923 | −8.877 | 1.000 | 2.29 |
| ATOM | 41 | C15 | 0 | 2.096 | 10.484 | −7.163 | 1.000 | 2.37 |
| ATOM | 42 | H15 | 0 | 1.930 | 11.215 | −7.778 | 1.000 | 2.84 |
| ATOM | 43 | H15 | 0 | 2.952 | 10.646 | −6.736 | 1.000 | 2.84 |
| ATOM | 44 | C16 | 0 | 0.717 | 5.784 | 2.444 | 1.000 | 2.97 |
| ATOM | 45 | H16 | 0 | 1.528 | 5.540 | 1.973 | 1.000 | 3.56 |
| ATOM | 46 | H16 | 0 | 0.195 | 4.978 | 2.582 | 1.000 | 3.56 |

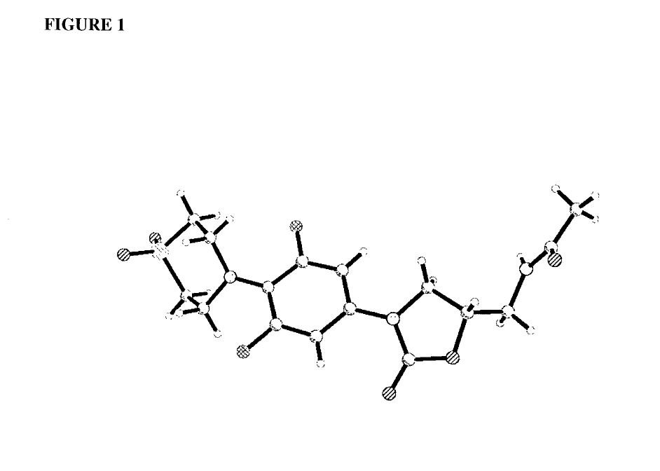

The three dimensional structure of the thiazine oxazolidinone is illustrated in FIG. 1 with a ball and stick model representing the crystal structure coordinates of anhydrous crystalline N-({(5S)-3-[4-(1,1-dioxido-4-thiomorpholinyl)-3,5-difluorophenyl]-2-oxo-1,3-oxazolidin-5-yl}-methyl)acetamide as listed in Table 1.

Powder X-ray Diffraction

Crystalline organic compounds consist of a large number of atoms that are arranged in a periodic array in three-dimensional space. The structural periodicity normally manifests distinct physical properties, such as sharp, explicit spectral features by most spectroscopic probes (e.g., X-ray diffraction, infrared and solid state NMR). X-ray diffraction (XRD) is acknowledged to be one of the most sensitive methods to determine the crystallinity of solids. Crystals yield explicit diffraction maxima which arise at specific angles consistent with the lattice interplanar spacings, as predicted by Bragg's law. On the contrary, amorphous materials do not possess long-range order. They often retain additional volume between molecules, as in the liquid state. Amorphous solids normally unveil a featureless XRD pattern with broad, diffuse halos because of the absence of the long range order of repeating crystal lattice.

Powder X-ray diffraction has been reportedly been used to characterize different crystal forms of organic compounds (e.g., compounds useful in pharmaceutical compositions). See, for example, U.S. Pat. Nos. 5,504,216 (Holohan et al), 5,721,359 (Dunn et al.), 5,910,588 (Wangnick et al.), 6,066,647 (Douglas et al.), 6,225,474 (Matsumoto et al.), 6,239,141 (Allen et al.), 6,251,355 (Murata et al.), 6,288,057 (Harkness), 6,316,672 (Stowell et al.), 6,329,364 (Groleau), and U.S. Pat. Application Publication Nos. 2001/0003752 (Talley et al.), 2002/0038021 (Barton et al.), and 2002/0045746 (Barton et al.).

Crystalline materials are preferred in many pharmaceutical applications. Crystalline forms are thermodynamically more stable than amorphous forms of the same substance. This thermodynamic stability is reflected in the lower solubility and improved physical stability of the crystalline form. The regular packing of the molecules in the crystalline solid denies the incorporation of chemical impurities. Hence crystalline materials generally possess higher chemical purity than their amorphous counterparts. The packing in the crystalline solid constrains the molecules to well defined lattice positions and reduces the molecular mobility that is the prerequisite for chemical reactions. Hence, crystalline solids, with very few notable exceptions, are chemically more stable than amorphous solids of the same molecular composition. Preferably, the crystalline forms of N-({(5S)-3-[4-(1,1-dioxido-4-thiomorpholinyl)-3,5-difluorophenyl]-2-oxo-1,3-oxazolidin-5-yl}methyl)acetamide disclosed in the present application possess one or more of the advantageous chemical and/or physical properties disclosed herein.

The crystalline forms of N-({(5S)-3-[4-(1,1-dioxido-4-thiomorpholinyl)-3,5-difluorophenyl]-2-oxo-1,3-oxazolidin-5-yl}methyl)acetamide disclosed in the present application have distinct powder X-ray diffraction profiles. For example, the anhydrous crystalline form of the thiazine oxazolidinone can be distinguished from the solvated forms of the thiazine oxazolidinone disclosed herein by the presence of characteristic diffraction peaks. Characteristic diffraction peaks as used herein are peaks selected from the most intense peaks of the observed diffraction pattern. Preferably, the characteristic peaks are selected from about 20 of the most intense peaks, more preferably from about 10 of the most intense peaks, and most preferably from about 5 of the most intense peaks in the diffraction pattern.

Preferably, an anhydrous crystalline N-({(5S)-3-[4-(1,1-dioxido-4-thiomorpholinyl)-3,5-difluorophenyl]-2-oxo-1,3-oxazolidin-5-yl}methyl)acetami de has characteristic diffraction peaks at about 21.9, 21,4, and 16.1 degrees two-theta, more preferably at about 29.5, 21.9, 21.4, 18.4, 16.1, and 9.2 degrees two-theta, and most preferably has the characteristic diffraction peaks as listed in Table 5.

Preferably, a solvated crystalline N-({(5S)-3-[4-(1,1-dioxido-4-thiomorpholinyl)-3,5-difluorophenyl]-2-oxo-1,3-oxazolidin-5-yl}methyl)acetami de has characteristic diffraction peaks at about 8.9 and 6.3 degrees two-theta, more preferably at about 19.2, 18.9, 16.4, 9.6, and 6.3 degrees two-theta, and most preferably has the characteristic diffraction peaks as listed in Table 2, Table 3, Table 4, or combinations thereof.

Methods of Isolation

In one embodiment, the anhydrous crystalline form of the thiazine oxazolidinone may be isolated by inducing crystallization of a solvated form of the thiazine oxazolidinone from an aqueous solution. Preferably the aqueous solution includes water and a water miscible organic solvent. Preferably the water miscible organic solvent is non-chlorinated. Useful water miscible organic solvents include, for example, tetrahydrofuran (THF), acetone, acetonitrile, and alcohols (e.g., methanol, ethanol, propanol, and isopropanol (IPA or 2-propanol)). Preferred water miscible organic solvents include, for example, tetrahydrofuran, acetone, ethanol, and methanol. More preferred water miscible organic solvents include, for example, tetrahydrofuran and methanol. The aqueous solution preferably includes a water miscible organic solvent, more preferably at least about 10% by weight water miscible organic solvent, and most preferably at least about 25% by weight water miscible organic solvent. The aqueous solution preferably includes at most about 50% by weight water miscible organic solvent, and more preferably at most about 33% by weight water miscible organic solvent.

The aqueous solution preferably includes at least about 2% of the thiazine oxazolidinone, and more preferably at least about 5% of the thiazine oxazolidinone. The aqueous solution preferably includes at most about 10% of the thiazine oxazolidinone, and more preferably at most about 6% of the thiazine oxazolidinone.

The anhydrous crystalline form of the thiazine oxazolidinone is preferably induced to crystallize by cooling the aqueous solution. Preferably the aqueous solution is initially at about the reflux temperature of the aqueous solution. Preferably the aqueous solution is cooled to a temperature of about 0° C. to 10° C. Preferably the cooling rate is maintained at a rate of at least about 2° C./hour, and more preferably at least about 5° C./hour. Preferably the cooling rate is maintained at a rate of at most about 20° C./hour, more preferably at most about 15° C./hour, and most preferably at most about 10° C./hour. The cooling rate does not need to be maintained at a constant rate throughout the cooling cycle, but can be varied as desired.

Optionally, seeding with crystals of the anhydrous form may be used if desired. If seed crystals are added to the aqueous solution, they are preferably added to the aqueous solution before precipitation has begun. More preferably the seed crystals are added at the meta-stable stage of the crystallization. The meta-stable stage of crystallization is defined as the stage at which the solution is saturated but crystallization has not yet begun.

In another embodiment, anhydrous crystalline N-({(5S)-3-[4-(1,1-dioxido-4-thiomorpholinyl)-3,5-difluorophenyl]-2-oxo-1,3-oxazolidin-5-yl}methyl)acetamide may be isolated by inducing crystallization of a solution of N-({(5S)-3-[4-(1,1-dioxido-4-thiomorpholinyl)-3,5-difluorophenyl]-2-oxo-1,3-oxazolidin-5-y}Imethyl)acetamide, wherein the solution includes methanol and methylene chloride. Preferably the solution includes at least about 2% by weight methanol. Preferably the solution includes at most about 5% by weight methanol. The crystallization may be induced by, for example, cooling the solution.

The anhydrous crystalline form preferably has a melting point of at least about 170° C., more preferably at least about 180° C., and most preferably at least about 187° C.

The anhydrous crystalline form is preferably not hygroscopic. Preferably, the anhydrous crystalline form picks up at most about 1% by weight moisture, more preferably at most about 0.5% by weight moisture, and most preferably at most about 0.3% by weight moisture at 90% relative humidity during a dynamic moisture sorption scan from 0 to 90% relative humidity at 25° C. using 3% humidity steps and an equilibrium time of not more than 180 minutes at each step.

The anhydrous crystalline form exhibits excellent bulk drug stability in aging tests. Preferably, the anhydrous crystalline form remains stable after storage at elevated temperatures and humidities. Preferably the anhydrous crystalline form remains stable at 56° C., and more preferably at 70° C., at 70% humidity for two months.

Solvated forms of the thiazoline oxazolidinone may be isolated by cooling a solution of the thiazine oxazolidinone in an organic solvent. Preferably, the solvate includes the organic solvent. Preferably, the organic solvent is non-halogenated. Preferred organic solvents include tetrahydrofuran, acetone, ethyl acetate, acetonitrile, and alcohols. Optionally, the solution may also include water. If water is included, the solution preferably includes at most about 5% by weight water. Preferably the solution includes at least about 2% by weight of the thiazine oxazolidinone.

Pharmaceutical Compositions

The pharmaceutical compositions of this invention may be prepared by combining the anhydrous crystalline form of the present invention with a solid or liquid pharmaceutically acceptable carrier and, optionally, with pharmaceutically acceptable adjuvants and excipients employing standard and conventional techniques. Solid form compositions include powders, tablets, dispersible granules, capsules, cachets and suppositories. A solid carrier can be at least one substance which may also function as a diluent, flavoring agent, solubilizer, lubricant, suspending agent, binder, tablet disintegrating agent, and encapsulating agent. Inert solid carriers include magnesium carbonate, magnesium stearate, talc, sugar, lactose, pectin, dextrin, starch, gelatin, cellulosic materials, low melting wax, cocoa butter, and the like. Liquid form compositions include solutions, suspensions and emulsions. For example, there may be provided solutions of the anhydrous crystalline form of this invention dissolved in water and water-propylene glycol and water-polyethylene glycol systems, optionally containing suitable conventional coloring agents, flavoring agents, stabilizers and thickening agents. Preferably, the pharmaceutical composition is provided employing conventional techniques in unit dosage form containing effective or appropriate amounts of the active component, that is, the compound of this invention.

The quantity of active component, that is the compound of this invention, in the pharmaceutical composition and unit dosage form thereof may be varied or adjusted widely depending upon the particular application, the potency of the particular compound and the desired concentration. Generally, the quantity of active component is about 0.5% by weight to about 90% by weight of the composition.

In therapeutic use for treating, or combating, bacterial infections in a mammal (i.e. human and animals) the compounds or its pharmaceutical compositions will be administered orally, topically, transdermally, and/or parenterally at a dosage to obtain and maintain a concentration which will be antibacterially effective. Preferably, such antibacterially effective amount of dosage of active component will be about 0.1 to about 100 mg/kg of body weight/day, and more preferably about 1.0 to about 50 mg/kg of body weight/day. It is to be understood that the dosages may vary depending upon the requirements of the patient and the severity of the bacterial infection being treated.

Also, it is to be understood that the initial dosage administered may be increased beyond the above upper level in order to rapidly achieve the desired blood-level or the initial dosage may be smaller than the optimum and the daily dosage may be progressively increased during the course of treatment depending on the particular situation. If desired, the daily dose may also be divided into multiple doses for administration, e.g., two to four times per day.

The compound according to this invention is administered parenterally, i.e., by injection, for example, by intravenous injection or by other parenteral routes of administration. Pharmaceutical compositions for parenteral administration will generally contain a pharmaceutically acceptable amount of the compound as a soluble salt (acid addition salt or base salt) dissolved in a pharmaceutically acceptable liquid carrier such as, for example, water-for-injection and a buffer to provide a suitably buffered isotonic solution, for example, having a pH of about 3.5 to about 6. Suitable buffering agents include, for example, trisodium orthophosphate, sodium bicarbonate, sodium citrate, N-methylglucamine, L(+)-lysine and L(+)-arginine to name but a few representative buffering agents. The compound of the invention generally will be dissolved in the carrier in an amount sufficient to provide a pharmaceutically acceptable injectable concentration of about 1 mg/ml to about 400 mg/ml of solution. The resulting liquid pharmaceutical composition will be administered so as to obtain the above-mentioned antibacterially effective amount of dosage. The compound of this invention is advantageously administered orally in solid and liquid dosage forms.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, (x-ketoglutarate, maleate, fumarate, benzenesulfonate and (X-glycerophosphate. Suitable inorganic salts may also be formed, including hydrobromide, hydrochloride, sulfate, nitrate, bicarbonate, and carbonate salts.

The present invention is illustrated by the following examples. It is to be understood that the particular examples, materials, amounts, and procedures are to be interpreted broadly in accordance with the scope and spirit of the invention as set forth herein.

### EXAMPLES

X-ray diffraction (XRD) patterns were measured on a Scintag X2 diffractometer (Thermo ARL, Ecublens, Switzerland) equipped with a theta-theta goniometer. Single crystal x-ray diffraction was carried out on a Bruker SMART 6K CCD Area Detector diffractometer (Bruker AXS, Karlsruhe, Germany). Mass spectrometry (MS) (ionization type) was carried out on a Micromass Platform II spectrometer (Micromass, Manchester, United Kingdom). High pressure liquid chromatography (HPLC) was carried out on an Agilent 1100 Series chromatograph (Agilent Technologies, Palo Alto, Calif.). Melting points were determined using a TA Instruments 2920 differential scanning calorimeter (TA Instruments, New Castle, Del.) module with a Thermal Analyst 5000 controller. Hygroscopicity was assessed by dynamic moisture sorption gravimetry (DMSG) using a Controlled Atmosphere Microbalance (Pharmacia Corp., Kalamazoo, Mich.). All chemicals used are available from Aldrich Chemical Co., Milwaukee, Wis., unless otherwise specified. N-({(5S)-3-[3,5-difluoro-4-(4-thiomorpholinyl)phenyl]-2-oxo-1,3-oxazolidin-5-yl}methyl)acetamide was prepared as described in PCT International Publication Number WO 01/98297 (Barbachyn et al.).

Methods

Melting Point Determination

Melting points were determined using a TA Instruments 2920 differential scanning calorimeter (TA Instruments, New Castle, Del.) module with a Thermal Analyst 5000 controller. Data were collected and analyzed using TA Instruments Thermal Solutions for NT Ver. 1.3L and Universal Analysis for NT Ver. 2.4F Samples of about 1 mg were accurately weighed into aluminum pans with lids (TA part numbers 900779 and 900786), which were crimped to ensure good thermal contact. The samples were evaluated using a linear heating ramp of 10° C./minute from ambient to approximately 300° C. The DSC cell was purged with a dry nitrogen flow of 50 standard cubic centimeters per minute (sccm).

Powder X-Ray Diffraction (XRD)

Powder X-Ray diffraction was performed using a Scintag X2 Advanced Diffraction System operating under Scintag DMS/NT 1.30a and Microsoft Windows NT 4.0 software. The system uses a Copper X-Ray source maintained at 45 kV and 40 MA to provide CuK(α₁ emission of 1.5406 Å and a solid-state Peltier cooled detector. The beam aperture was controlled using tube divergence and anti-scatter slits of 2 and 4 mm and detector anti-scatter and receiving slits of 0.5 and 0.2 mm width. Data was collected from 2 to 35° two-theta using a step scan of 0.03°/point with a one second per step counting time. Scintag round, top loading stainless steel sample cups with 12 mm diameter inserts were utilized for the experiments. Bulk drug was sampled as-is and placed into the sample tray without any preparation. Some specific samples were also hand-ground in a mortar and pestle before they were run. Data analysis was completed using Origin 6.0 (Microcal Software, Northampton Mass.).

Dynamic Moisture Sorption Gravimetry (DMSG)

DMSG isotherms were collected on the variable temperature controlled atmospheric microbalance. Approximately 10 mg samples were used in the balance. Samples were run as received. The humidity was sequentially set between 0 and 90% RH in 3% RH steps. The mass was then measured every two minutes. The RH was changed to the next value when the mass of the sample was stable to within 0.5 μg in 480 seconds. A Visual Basic program was used to control the data collection and export the information to an Excel spreadsheet.

Thermal Analysis

Differential Scanning Calorimetry (DSC) data was obtained by crimping the powdered sample into an aluminum DSC pan. Samples were run as received, sizes were about 1 mg. Temperatures were typically scanned to 320° C. at a scan rate of 10° C. per minute. The DSC was a TA Instruments 2920 calorimeter. The data analysis software used was TA's Universal Analysis V 1. 1 OB.

### Example 1

### Preparation of N-({(5S)-3-[4-(1,1-dioxido-4-thiomnorpholinyl)-3,5-difluorophenyl]-2-oxo-1,3-oxazolidin-5-yl}methyl) acetamide.

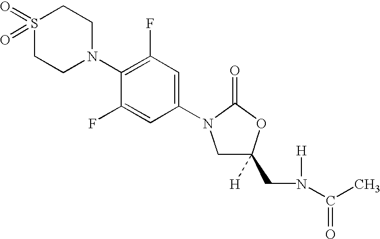

N-({(5S)-3-[3,5-difluoro-4-(4-thiomorpholinyl)phenyl]-2-oxo-1,3-oxazolidin-5-yl}methyl)acetamide (4.17 g, 11.22 mmol) was dissolved in a solution of 25% water/acetone and then treated sequentially with 4-methylmorpholine N-oxide (3 eq, 3.94 g, 33.66 mmol) and a catalytic amount of osmium tetroxide (0.9 mL). The reaction was stirred for 60 hours at room temperature under nitrogen. At that point, another 2 mL of osmium tetroxide and another 1 eq of 4-methylmorpholine N-oxide were added. After 4 hours, the reaction was then quenched with a solution of sodium hydrosulfite and then extracted with methylene chloride three times. The organic layer was washed with sodium hydrosulfite and brine, dried over sodium sulfate, filtered, and concentrated. The crude product was then purified on silica gel and concentrated in 2-5% methanol in methylene chloride solution to yield 1.7 g as lot 1, and 1.9 g as lot 2, in a total of 3.6 g (80%) of the title compound as a white solid, m.p. of lot 1: 188-190° C. (measured by DSC).

MS (ESI+) for m/z 404 (M+H)⁺, MS (ESI+) for m/z 426 (M+Na)⁺, MS (ESI−) for m/z 402 (M−H)⁻.

### Example 2

### Isolation of the 2-Propanol Solvate of the Thiazine Oxazolidinone

N-({(5S)-3-[4-(1,1-dioxido-4-thiomorpholinyl)-3,5-difluorophenyl]-2-oxo-1, 3-oxazolidin-5-yl}methyl)acetamide (0.5 g) was dissolved in 14 mL of refluxing 2-propanol. The mixture was then cooled to 0° C. and filtered. The filtered solids were dried to give 0.46 g of the 2-propanol solvate of the thiazine oxazolidinone.

X-ray powder diffraction of the 2-propanol solvate of the thiazine oxazolidinone gave the pattern listed in Table 2.

TABLE 2
Powder X-ray Diffraction of Isopropanol Solvate
Peak listing from 2 to 30 degrees two-theta

| Peak Position | | Approx. Relative |
|---|---|---|
| d-Spacing | | Intensity |
| (Angstroms) | Degrees Two-theta* | I/Io (%) |
| 14.07 | 6.28 | 53 |
| 9.19 | 9.61 | 23 |
| 7.05 | 12.54 | 5 |
| 5.44 | 16.29 | 30 |
| 4.71 | 18.83 | 100 |
| 4.65 | 19.07 | 16 |
| 4.35 | 20.42 | 13 |
| 4.11 | 21.63 | 7 |
| 4.05 | 21.92 | 8 |
| 3.94 | 22.54 | 12 |
| 3.86 | 23.04 | 6 |
| 3.10 | 28.76 | 8 |

*Peak positions using Cu KL3 radiation at 1.54056 angstroms

### Example 3

### Isolation of the Ethyl Acetate Solvate of the Thiazine Oxazolidinone

5 N-({(5S)-3-[4-(1,1-dioxido-4-thiomorpholinyl)-3,5-difluorophenyl]-2-oxo-1,3-oxazolidin-5-yl}methyl)acetamide (0.5 g) was dissolved in 26 mL of refluxing ethyl acetate. The mixture was then cooled to 0° C. and filtered. The filtered solids were dried to give 0.38 g of the ethyl acetate solvate of the thiazine oxazolidinone.

X-ray powder diffraction of the ethyl acetate solvate of the thiazine oxazolidinone gave the pattern listed in Table 3.

TABLE 3
Powder X-ray Diffraction of Ethyl Acetate Solvate
Peak listing from 2 to 30 degrees two-theta

| Peak Position | | Approx. Relative |
|---|---|---|
| d-Spacing | | Intensity |
| (Angstroms) | Degrees Two-theta* | I/Io (%) |
| 13.87 | 6.37 | 43 |
| 9.09 | 9.72 | 14 |
| 6.99 | 12.66 | 6 |
| 5.40 | 16.41 | 26 |
| 4.67 | 18.99 | 100 |
| 4.63 | 19.16 | 18 |
| 4.33 | 20.49 | 15 |
| 4.08 | 21.78 | 9 |
| 3.92 | 22.66 | 11 |
| 3.82 | 23.26 | 7 |
| 3.09 | 28.88 | 6 |

*Peak positions using Cu KL3 radiation at 1.54056 angstroms

### Example 4

### Isolation of the Tetrahydrofuran Solvate of the Thiazine Oxazolidinone

5 N-({(5S)-3-[4-(1,1-dioxido-4-thiomorpholinyl)-3,5-difluorophenyl]-2-oxo-1,3-oxazolidin-5-yl}methyl)acetamide (8.5 g) was dissolved in a refluxing mixture of 100 mL of tetrahydrofuran and 15 mL of water. The mixture was then cooled to 0° C. and filtered. The filtered solids were dried to give 6.2 g of the tetrahydrofuran solvate of the thiazine oxazolidinone.

X-ray powder diffraction of the tetrahydrofuran solvate of the thiazine oxazolidinone gave the pattern listed in Table 4.

TABLE 4
Powder X-ray Diffraction of Tetrahydrofuran Solvate
Peak listing from 2 to 30 degrees two-theta

| Peak Position | | Approx. Relative |
|---|---|---|
| d-Spacing | | Intensity |
| (Angstroms) | Degrees Two-theta* | I/Io (%) |
| 14.11 | 6.26 | 30 |
| 9.32 | 9.49 | 15 |
| 5.41 | 16.36 | 22 |
| 4.72 | 18.80 | 100 |
| 4.60 | 19.30 | 15 |
| 4.32 | 20.52 | 7 |
| 4.19 | 21.20 | 7 |
| 4.08 | 21.76 | 8 |
| 3.93 | 22.60 | 10 |
| 3.09 | 28.85 | 5 |

*Peak positions using Cu KL3 radiation at 1.54056 angstroms

### Example 5

### Isolation of the Anhydrous Crystalline Thiazine Oxazolidinone

Procedure A: The 2-propanol solvate as prepared in Example 2 (12.7 g, approximately 6% by weight 2-propanol content) was dissolved in 250 mL of water at 95-100° C. The mixture was cooled at approximately 5° C./hour from 100° C. to 80° C. and at 10° C./hour from 80° C. to 5° C. The mixture was then filtered, and the filtered solid was dried to give 12.1 g of anhydrous crystalline thiazine oxazolidinone.

Procedure B: In THF/water: The 2-propanol solvate as prepared in Example 2 (10 g, approximately 6% by weight 2-propanol content) was dissolved in 50 ml of a liquid containing 25% by volume water and 75% by volume tetrahydrofuran. The above solution was added to 200 mL of water at a rate of 0.8 mL/min at 20-25° C. An agitation rate of 250 rpm or greater was used during the addition. When addition was complete, the mixture was cooled to 0 to 5° C. and then filtered. The filtered solid was dried to give 8.2 g of anhydrous crystalline thiazine oxazolidinone.

Procedure C: A methylene chloride solution of the thiazine oxazolidinone (about 7 kg) was distilled at a pressure of 740 to 780 mm Hg while water and methanol were added. The distillation was continued until the vapor temperature reached 85 to 87° C. The final volume was 120 to 160 L. The mixture was then cooled gradually until a temperature of 60 to 62° C. was reached, seeded with 35 g of anhydrous thiazine oxazolidinone crystals. When the mixture was at a temperature of 58 to 60° C., 35 g of anhydrous thiazine oxazolidinone crystals were again added. The mixture was then cooled at a rate of 5° C./hour to a temperature of 0 to 5° C. The slurry was filtered, and the filtered solid was dried to give 5.5 kg of anhydrous crystalline thiazine oxazolidinone.

X-ray powder diffraction of the anhydrous crystalline thiazine oxazolidinone gave the pattern listed in Table 5.

TABLE 5
Powder X-ray Diffraction of Anhyhdrous Crystalline Thiazine
Oxazolidinone
Peak listing from 2 to 30 degrees two-theta

| Peak Position | | Approx. Relative |
|---|---|---|
| d-Spacing | | Intensity |
| (Angstroms) | Degrees Two-theta* | I/Io (%) |
| 9.64 | 9.17 | 32 |
| 5.49 | 16.13 | 78 |
| 5.21 | 17.01 | 9 |
| 4.82 | 18.38 | 67 |
| 4.67 | 18.97 | 17 |
| 4.50 | 19.72 | 8 |
| 4.37 | 20.28 | 13 |
| 4.32 | 20.55 | 11 |
| 4.24 | 20.92 | 14 |
| 4.16 | 21.36 | 83 |
| 4.05 | 21.95 | 100 |
| 3.76 | 23.67 | 15 |
| 3.39 | 26.30 | 16 |
| 3.22 | 27.71 | 10 |
| 3.02 | 29.51 | 17 |
| 2.62 | 34.15 | 7 |
| 2.61 | 34.29 | 5 |
| 2.50 | 35.89 | 7 |

*Peak positions using Cu KL3 radiation at 1.54056 angstroms

Hygroscopicity of the anhydrous crystalline thiazine oxazolidinone was assessed at 25° C. and indicated that the material was not hygroscopic, picking up approximately 0.2% moisture during a dynamic moisture sorption scan from 0 to 87% relative humidity using 3% humidity steps and an equilibrium time of not more than 180 minutes at each step.

### Example 6

### Bulk Drug Stability

Aliquots (about 0.3 mg) of bulk anhydrous crystalline N-({(5S)-3-[4-(1,1-dioxido-4-thiomorpholinyl)-3,5-difluorophenyl]-2-oxo-1,3-oxazolidin-5-yl}methyl)acetamide were weighed into 2-mL vials. The uncapped vials were placed either in a 75% relative humidity chamber (desiccator containing water saturated with sodium chloride) or in a dry chamber (desiccator containing Drierite). The chambers were placed in temperature controlled cabinets at 56° C. and 70° C. A single vial was removed from each desiccator at selected time points and was assayed using high pressure liquid chromatography (HPLC). The results are summarized in Table 6.

TABLE 6
Bulk Drug Stability of the Anhydrous Crystalline Form Measured as
% of Initial HPLC Peak Area.

| Storage Conditions | Day 6 | Day 13 | Day 27 | Day 61 |
|---|---|---|---|---|
| 56° C., dessicator | 100.1 | 99.4 | 100.0 | 99.8 |
| 56° C., | 100.1 | 99.3 | 99.9 | 100.1 |
| 75% relative humidity | | | | |
| 70° C., dessicator | 100.1 | 99.7 | 100.0 | 100.0 |
| 70° C., | 100.1 | 99.6 | 99.9 | 99.9 |
| 75% relative humidity | | | | |

### Example 7

### Single Crystal X-Ray Diffraction

An anhydrous crystal of N-({(5S)-3-[3,5-difluoro-4-(4-thiomorpholinyl)phenyl]-2-oxo-1,3-oxazolidin-5-yl}methyl)acetamide was grown in dichloromethane at 25° C., with an approximate size of 0.025×0.02×0.2 mm, diffracted to 0.84 Å. The structure was solved by standard methods known in the art. The crystal contained one molecule per asymmetric unit with cell dimensions of a=8.7790 Å, b=11.4911 Å, c=17.4233 Å, α=β=γ=90° in orthorhombic space group symmetry P2₁2₁2₁.

The complete disclosure of all patents, patent applications, and publications, and electronically available material (e.g., GenBank amino acid and nucleotide sequence submissions) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. An anhydrous crystal comprising N-({(5S)-3-[4-(1,1-dioxido-4-thiomorpholinyl)-3, 5-difluorophenyl]-2-oxo-1,3-oxazolidin-5-yl}methyl) acetamide.

2. An anhydrous crystal comprising a thiazine oxazolidinone having the structure:

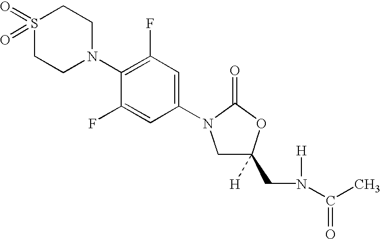

3. The anhydrous crystal of claim 2 having a melting point of at least about 170° C.

4. The anhydrous crystal of claim 3 having a melting point of at least about 180° C.

5. The anhydrous crystal of claim 4 having a melting point of at least about 187° C.

6. The anhydrous crystal of claim 2 wherein the thiazine oxazolidinone is stable in a bulk drug stability test at 56° C. and 75% relative humidity for at least 25 days.

7. The anhydrous crystal of claim 2 wherein the thiazine oxazolidinone is stable in a bulk drug stability test at 70° C. and 75% relative humidity for at least 60 days.

8. A crystal comprising N-({(5S)-3-[4-(1,1-dioxido-4-thiomorpholinyl)-3, 5-difluorophenyl]-2-oxo-1,3-oxazolidin-5-yl}methyl)acetamide, wherein the crystal further comprises at most about 1% by weight water.

9. The crystal of claim 8 comprising at most about 0.5% by weight water.

10. The crystal of claim 9 comprising at most about 0.1% by weight water.

11. The crystal of claim 8 wherein the N-({(5S)-3-[4-(1,1-dioxido-4-thiomorpholinyl)-3, 5-difluorophenyl]-2-oxo-1,3-oxazolidin-5-yl}methyl)acetamide is stable in a bulk drug stability test at 56° C. and 75% relative humidity for at least 25 days.

12. The crystal of claim 8 wherein the N-({(5S)-3-[4-(1,1-dioxido-4-thiomorpholinyl)-3, 5-difluorophenyl]-2-oxo-1,3-oxazolidin-5-yl}methyl)acetamide is stable in a bulk drug stability test at 70° C. and 75% relative humidity for at least 60 days.

13. A crystal comprising N-({(5S)-3-[4-(1,1-dioxido-4-thiomorpholinyl)-3, 5-difluorophenyl]-2-oxo-1,3-oxazolidin-5-yl}methyl) acetamide having orthorhombic space group symmetry P2₁2₁2₁.

14. A crystal comprising N-({(5S)-3-[4-(1,1-dioxido-4-thiomorpholinyl)-3, 5-difluorophenyl]-2-oxo-1,3-oxazolidin-5-yl}methyl) acetamide comprising a unit cell defined by the dimensions a, b, c, α, β, and γ, wherein a is about 8.8 Å, b is about 11.5 Å, c is about 17.4 Å, and α=β=γ=90°.

15. A crystal comprising N-({(5S)-3-[4-(1,1-dioxido-4-thiomorpholinyl)-3, 5-difluorophenyl]-2-oxo-1,3-oxazolidin-5-yl}methyl) acetamide having orthorhombic space group symmetry P2₁2₁2₁ and comprising a unit cell defined by the dimensions a, b, c, α, β, and γ, wherein a is about 8.8 Å, b is about 11.5 Å, c is about 17.4 Å, and α=β=γ=90°.

16. A crystal comprising N-({(5S)-3-[4-(1,1-dioxido-4-thiomorpholinyl)-3, 5-difluorophenyl]-2-oxo-1,3-oxazolidin-5-yl}methyl) acetamide, wherein the atomic positions of the atoms of the N-({(5S)-3-[4-(1, 1-dioxido-4thiomorpholinyl)-3,5-difluorophenyl]-2-oxo-1,3-oxazolidin-5-yl}methyl)acetamide are defined by a set of points having a root mean square deviation of less than about 0.1 Å from the structure coordinates listed in Table 1.

17. The crystal of claim 16 wherein the atomic positions of the atoms are defined by the structure coordinates listed in Table 1.

18. A crystal comprising N-({(5S)-3-[4-(1,1-dioxido-4-thiomorpholinyl)-3, 5-difluorophenyl]-2-oxo-1,3-oxazolidin-5-yl}methyl) acetamide having characteristic diffraction peaks at about 21.9, 21,4, and 16.1 degrees two-theta in an X-ray powder diffraction pattern.

19. The crystal of claim 18 having characteristic diffraction peaks at about 29.5, 21.9, 21.4, 18.4, 16.1, and 9.2 degrees two-theta in an X-ray powder diffraction pattern.

20. The crystal of claim 19 having the characteristic diffraction peaks in an X-ray powder diffraction pattern as listed in Table 5.

21. A solvated crystal comprising N-({(5S)-3-[4-(1,1-dioxido-4-thiomorpholinyl)-3, 5-difluorophenyl]-2-oxo-1,3-oxazolidin-5-yl}methyl) acetamide, wherein the solvate comprises a solvent selected from the group consisting of tetrahydrofuran, acetone, ethyl acetate, acetonitrile, methanol, ethanol, propanol, isopropanol, and combinations thereof.

22. A crystal comprising N-({(5S)-3-[4-(1,1-dioxido-4-thiomorpholinyl)-3, 5-difluorophenyl]-2-oxo-1,3-oxazolidin-5-yl}methyl) acetamide having characteristic diffraction peaks at about 18.9 and 6.3 degrees two-theta in an X-ray powder diffraction pattern.

23. The crystal of claim 22 having characteristic diffraction peaks at about 19.2, 18.9, 16.4, 9.6, and 6.3 degrees two-theta in an X-ray powder diffraction pattern.

24. The crystal of claim 23 having the characteristic peaks in an X-ray powder diffraction pattern as listed in Table 2, Table 3, Table 4, or combinations thereof.

25. A method of isolating an anhydrous crystal comprising a thiazoline oxazolidinone, the method comprising cooling an aqueous solution of a solvate comprising N-({(5S)-3-[4-(1,1-dioxido-4-thiomorpholinyl)-3,5-difluorophenyl]-2-oxo-1, 3-oxazolidin-5-yl}methyl)acetamide to give the anhydrous crystal comprising the thiazoline oxazolidinone.

26. The method of claim 25 wherein the solvate comprises an organic solvent.

27. The method of claim 26 wherein the solvent is non-halogenated.

28. The method of claim 25 wherein the solvate comprises a solvent selected from the group consisting of tetrahydrofuran, acetone, ethyl acetate, acetonitrile, and alcohols.

29. The method of claim 25 wherein the aqueous solution further comprises a water miscible organic solvent.

30. The method of claim 29 wherein the water miscible organic solvent is non-halogenated.

31. The method of claim 29 wherein the water miscible organic solvent is selected from the group consisting of tetrahydrofuran, acetone, ethyl acetate, acetonitrile, and alcohols.

32. The method of claim 29 wherein the aqueous solution comprises at most about 50% by weight water miscible organic solvent.

33. The method of claim 29 wherein the aqueous solution comprises at most about 33% by weight water miscible organic solvent.

34. The method of claim 25 wherein the aqueous solution comprises at least about 2% by weight of the solvate of the thiazine oxazolidinone.

35. A method of isolating a solvated thiazoline oxazolidinone comprising cooling a composition comprising N-({(5S)-3-[4-(1,1-dioxido-4-thiomorpholinyl)-3, 5-difluorophenyl]-2-oxo-1,3-oxazolidin-5-yl}-methyl)acetamide and an organic solvent selected from the group consisting of tetrahydrofuran, acetone, ethyl acetate, acetonitrile, methanol, ethanol, propanol, isopropanol, and combinations thereof, to give the solvated thiazoline oxazolidinone.

36. The method of claim 35 wherein the medium further comprises water.

37. The method of claim 36 wherein the medium comprises at most about 50% by weight water.

38. The method of claim 35 wherein the medium comprises at least about 2% by weight of the thiazine oxazolidinone.

39. The method of claim 35 wherein the medium is a liquid.

40. The method of claim 39 wherein the thiazoline oxazolidinone is dissolved in the organic solvent.

41. A method of isolating an anhydrous crystal comprising N-({(5S)-3-[4-(1,1-dioxido-4-thiomorpholinyl)-3,5-difluorophenyl]-2-oxo-1,3-oxazolidin-5-yl}methyl)acetamide, the method comprising inducing crystallization of N-({(5S)-3-[4-(1,1-dioxido-4-thiomorpholinyl)-3,5-difluorophenyl]-2-oxo-1,3-oxazolidin-5-yl}methyl)acetamide from a medium comprising methanol and methylene chloride.

42. The method of claim 41 wherein the medium comprises at least about 2% by weight methanol.

43. The method of claim 41 wherein the medium comprises at most about 5% by weight methanol.
